(19) United States
(12) Patent Application Publication
(10) Pub. No.: US 2009/0105608 A1
(43) Pub. Date: Apr. 23, 2009

(54) Skin Biopsy Clamp

(76) Inventors: Kwok Wai Chiu, Plano, TX (US); Lawrence Chiu, Plano, TX (US); Timothy Chiu, Plano, TX (US); Kwok Hung Chung, Plano, TX (US)

Correspondence Address: Kwok Wai Chiu, Suite 105, 1220 Coit Road, Plano, TX, 75075, US

(21) Appl. No.: 11/875,938

(22) Filed: Oct. 21, 2007

(51) Int. Cl. A61B 10/02

(52) U.S. Cl. 600/564

(57) ABSTRACT

A spoon-shaped skin biopsy instrument is used in the removal of superficial skin samples and skin abnormalities for subsequent laboratory analysis. The spoon-shaped skin biopsy instrument is comprised of a pair of spoon-shaped cutting edges, which surround the desired skin sample, a crossed-members apparatus with finger loops, and a pivot point that connects the crossed members. This present invention maximizes surgical efficiency and handling and minimizes operation time.

### FIELD OF THE INVENTION

The present invention generally relates to the field of surgical instruments and a method using the same. In particular, the invention relates to a biopsy instrument for use in a biopsy surgery.

### BACKGROUND OF THE INVENTION

Skin biopsy is an essential test for diagnosing specific skin disorders. Basically, skin biopsy is a procedure that typically requires a dermatologist or family physician to excise a sample of skin for subsequent laboratory analysis. There are many types of skin biopsies including, but not limited to, shave/scoop biopsy, scalpel biopsy, and, most commonly, punch biopsy.

Punch biopsy is the most commonly performed skin biopsy and require minimal surgical skills, even for the most novice physician. In a punch biopsy, a small cylindrical punch, which consists of a single, solid, tubular handle with a variable circular cutting edge on the top, is screwed into the skin lesion through the full thickness of the skin tissue. The skin sample is then lifted above the incision using a 21-gauge needle, so as to prevent destruction of the sample, and excised at a level below the dermis. The same needle is then used to transport the sample into a preservative solution, which is then sent to the laboratory for analysis. The entire procedure is relatively short in duration and is performed under local anesthesia. Also, small punch biopsies, where the incision is less than 4 mm in diameter, typically do not require the use of sutures, while larger incisions from 5-8 mm in diameter usually do.

Although the punch biopsy is a relatively simple surgical procedure, there exist many disadvantages related to the current punch biopsy instrument. First, current punch biopsy instruments are comprised of only a single, tubular handle, which frequently becomes difficult to rotate as the skin sample size increases. Second, and most importantly, because the current punch biopsy procedure requires the use of a number of additional instruments (i.e., 21-gauge needle, scissors, etc.), problems may arise at any stage in the procedure and compromise successful excision and transportation (to the preservative solution) of the skin sample.

Therefore, a need exists for a simpler, more “user-friendly” punch biopsy instrument that combats and resolves the problems associated with current punch biopsy instruments.

### BRIEF SUMMARY OF THE INVENTION

Objectives and advantages of the current invention are presented below.

The primary purpose of the present invention is to offer an alternative, spoon-shaped skin biopsy instrument that will provide for increased efficiency, better handling, and reduced surgical time.

According to the present invention, a spoon-shaped skin biopsy instrument is comprised of a pair of spoon-shaped clamps measured to a size that is capable of excising skin samples of up to 4 mm in diameter, a crossed-members apparatus with finger loops, and a pivot point pen to connect the crossed members. This invention helps minimize operation time and maximizes surgical handling and efficiency.

### BRIEF DESCRIPTION OF THE DRAWINGS

The unique and novel characteristics of the present invention are set forth in the accompanying claims. In order to fully understand the present invention, its proper and preferred mode of use, objectives, and advantages, it is best to reference the following detailed descriptions of the illustrative embodiments when reading in conjunction with the accompanying drawings, wherein:

FIG. 1 is a ¾ profile view of the spoon-shaped skin biopsy instrument as used during the skin biopsy procedure in accordance with the present invention.

FIG. 2 is a side view of the spoon-shaped skin biopsy instrument in accordance with the present invention.

FIG. 3 is a ¾ profile view of the spoon-shaped apparatuses of the spoon-shaped skin biopsy instrument.

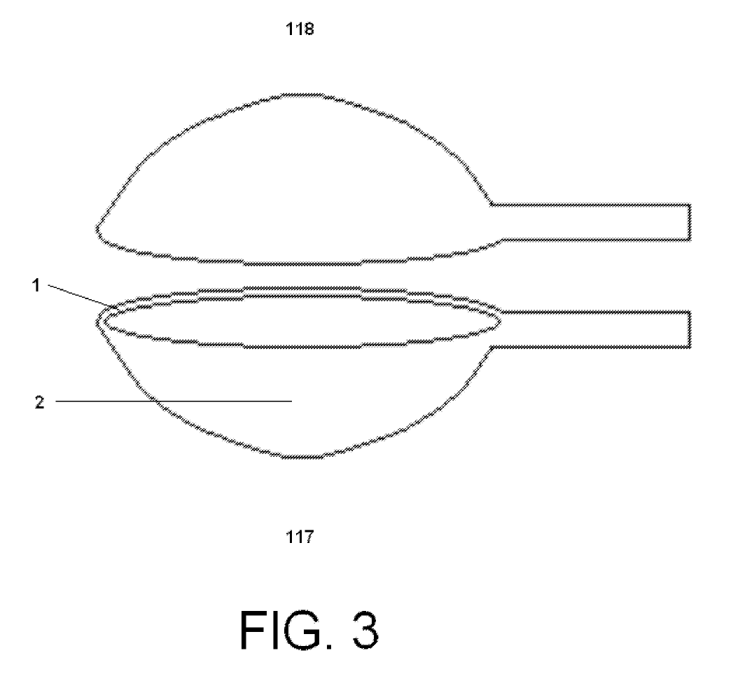

FIG. 4 is a side view of a preferred embodiment consisting of three sets of angular facets 13, 14, and 15 appearing on the side of each clamping tab.

FIG. 5 is a ¾ profile view of the spoon-shaped skin biopsy instrument in accordance with the present invention as it is being prepared to incise around the desired skin sample, which is marked with pen.

FIG. 6 is a ¾ profile view of the spoon-shaped skin biopsy instrument in use, incising the desired skin sample.

FIG. 7 is a side view of the spoon-shaped skin biopsy instrument in use, as it is incising into the desired skin sample.

FIG. 8 is a ¾ profile view of the spoon-shaped skin biopsy instrument in use, removing the desired skin sample.

FIG. 9 is a side view of the spoon-shaped skin biopsy instrument in use, as it is removing the desired skin sample.

FIG. 10 is a top view of the skin after the desired skin sample has been successfully removed.

### DETAILED DESCRIPTION OF THE INVENTION

Although the present invention described below is in reference to an ideal, preferred embodiment, it is recognized that other embodiments are possible. The concepts described below apply equally to other instruments for removing samples of other tissues, organs, organic matter, and can be applied to inorganic matter as well, so long as they follow the purpose of the teachings described herein.

Whereas many prior punch biopsy procedures require the utilization of multiple surgical instruments, the present invention allows one to carefully and precisely remove the desired skin sample with the use of a specialized spoon-shaped skin biopsy instrument that removes the skin sample and directly transports it to a preservative solution for laboratory analysis, thereby enhancing surgical efficiency by minimizing the surgery time and the potential mistakes that may arise with transportation and handling of the removed skin sample. Such efficient excision of the skin sample is made possible with the present spoon-shaped skin biopsy instrument, disclosed herein.

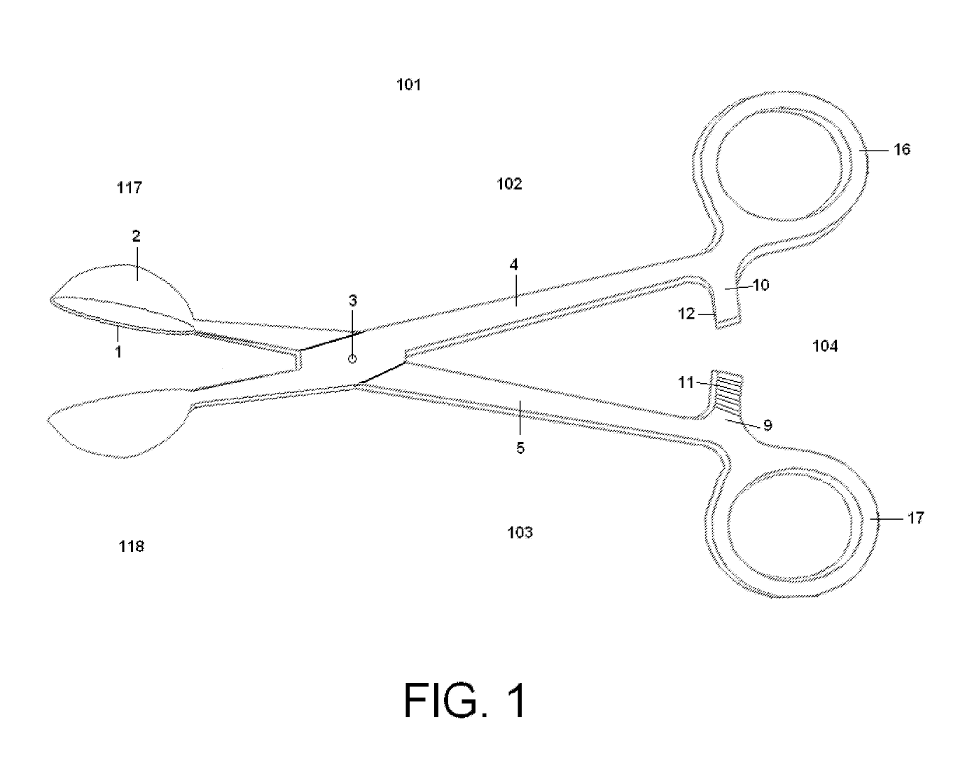

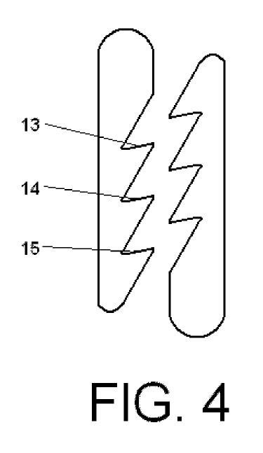

Referring to FIG. 1, the spoon-shaped skin biopsy instrument is generally referred to as numeral 101. According to the present invention, the spoon-shaped skin biopsy instrument 101 has: a pair of intersecting crossed members 102 and 103 and a pivot point 3 fixed between the crossed members. The pivot point 3 allows for movement of crossed members 102 and 103 toward and away from each other upon a common plane. The crossed members 102 and 103 consist of two handles 4 and 5 with finger loops 16 and 17 located at their mesial ends. Clamping tabs 9 and 10 extend distally from each finger loop 16 and 17 and are situated perpendicularly to the distal ends of crossed members 4 and 5 in such a way that the clamping tabs 9 and 10 will bind to each other using the angular facets 11 and 12 as the handles 4 and 5 are brought together and closed 104. The spoon-shaped apparatuses 117 and 118 are located on the distal end of the crossed members 102 and 103. The cutting edge 1 and spoon-shaped skin sample transporter 2 are located on each spoon-shaped apparatus 117 and 118. Referring to FIG. 4, the clamping teeth 13, 14, 15 are located on each of the angular facets 11 and 12 of clamping tabs 9 and 10.

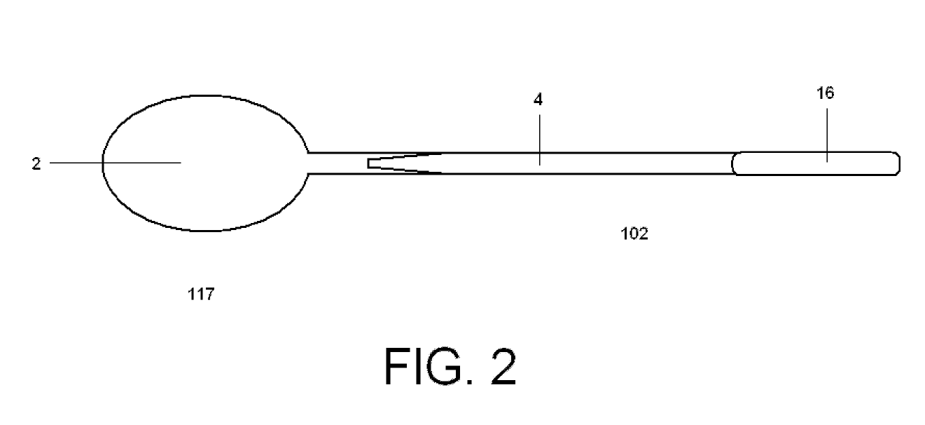

Referring to FIG. 2, a side view of the spoons-shaped skin-biopsy instrument 101 is shown. Most prominent is the spoon-shaped apparatus 117, the cutting edge 1, and the spoon-shaped skin sample transporter 2. Each cutting edge is situated on the distal ends of each spoon-shaped skin sample transporter. This is the most essential part of the present invention, as it allows for the combined actions of incising, cutting, extracting, and transporting of the desired skin sample in one efficient motion.

In a preferred embodiment of the present invention, the dimensions of the spoon-shaped skin-biopsy instrument 101 are as follows. Referring to FIG. 1, the curvature of each of the spoon-shaped apparatuses 117 and 118 resembles half of a hollow sphere, with its internal area and area of its outside surface sufficient enough to extract and transport a desired skin sample of up to 4 mm in diameter. The width and height of each of the spoon-shaped apparatuses 117 and 118 follow the same aforementioned conditions. The length of the cutting edge 1 will ultimately be determined by how large the spoon-shaped skin sample transporter 2 is and will line the distal edges of both spoon-shaped skin sample transporters 117 and 118. Referring to FIG. 1, the length of each crossed member 102 and 103 is variable, determined by need.

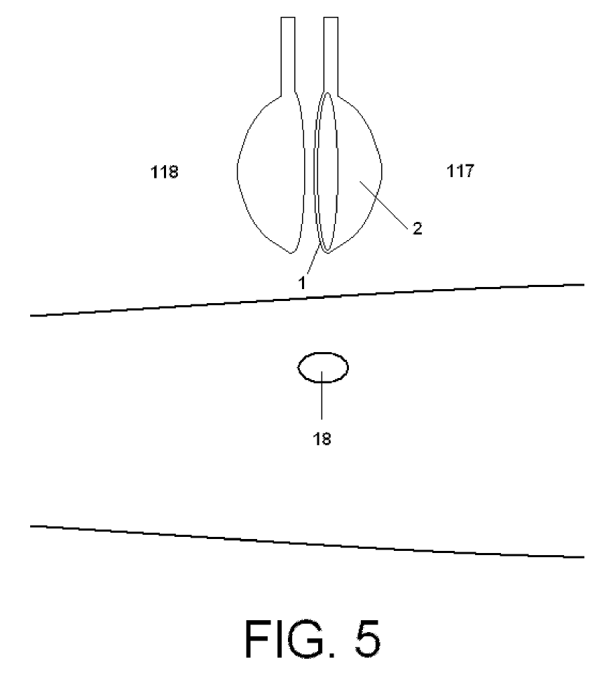

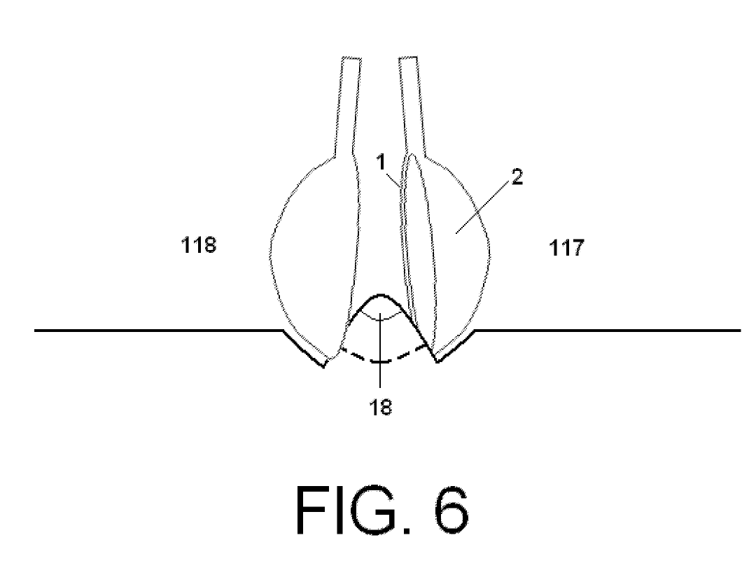

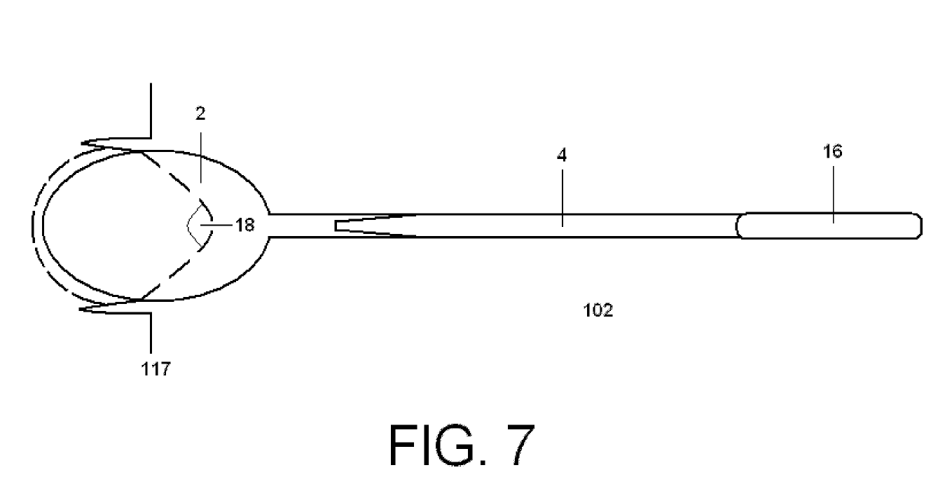

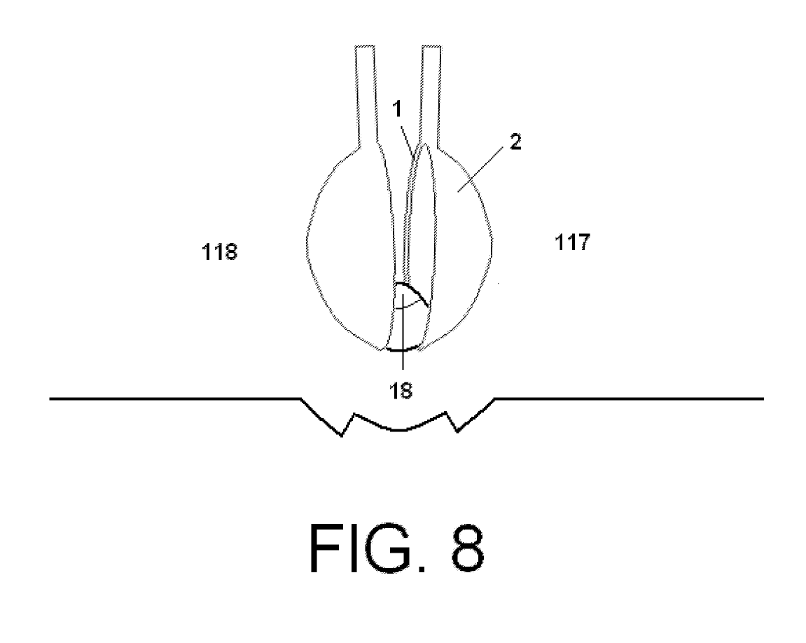

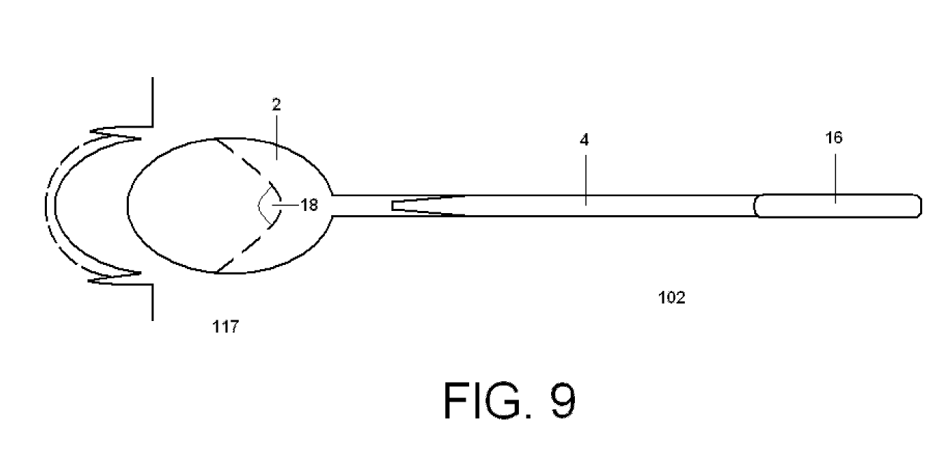

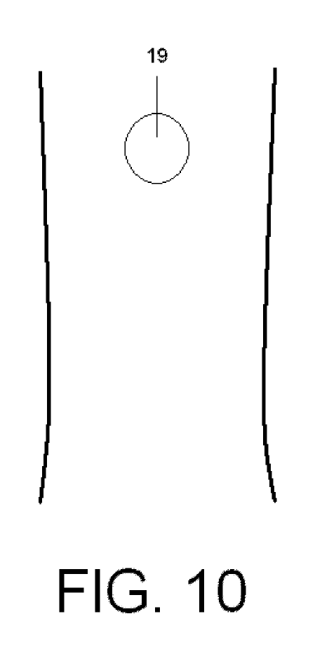

In practice, the selected area of skin is cleaned with the appropriate sterilizing solution and then anesthetized. Referring to FIG. 5, the spoon-shaped skin biopsy instrument 101, and specifically, the spoon-shaped apparatuses 117 and 118, is prepared to be used around the selected area of skin where the desired skin sample 18 is to be removed. Referring to FIG. 6, the spoon-shaped apparatuses 117 and 118 is used to clamp the area of skin surrounding the desired skin sample 18. Referring to FIG. 7, a side view of the spoon-shaped apparatus 117 is provided as it is clamped around and cutting into the desired skin sample. Referring to FIG. 8, the spoon-shaped apparatuses 117 and 118 are squeezed towards each other to a point where the desired skin sample 18 is cut out and removed from the skin. The desired skin sample 18, which is now enclosed by the spoon-shaped skin sample transporter 2, is then transported to a preservative solution. Referring to FIG. 9, a side view of the desired skin sample 18 is provided as it is removed from the skin and transported. Referring to FIG. 10, the skin biopsy procedure is complete when the desired skin sample 18 is removed and the area of skin where it was removed from is properly cleaned.

The spoon-shaped skin biopsy instrument 101 may be made of any material deemed suitable for use in a skin biopsy surgical procedure. In one possible embodiment of the present invention, stainless steel allow is used to construct the spoon-shaped skin biopsy instrument 101.

While the present invention has been illustrated and described in reference to a preferred embodiment, it is acknowledged by those proficient in performing skin biopsy surgical procedures that changes in form and detail may be made therein without departing from the purpose and scope of the present invention.

1. A spoon-shaped skin biopsy instrument for use in the removal of a desired skin sample, said spoon-shaped skin biopsy instrument comprising:
A pair of crossed, intersecting members having proximal and distal ends; and
A pivot pin placed in between the crossed members allowing for movement of the crossed members toward and away from each other;
The crossed members comprising
Handles with finger loops at the proximal ends of the crossed members;
Clamping tabs extending from the finger loops that overlap when the handles are closed, and wherein the angular facets, located on the clamping tabs, allow for the clamping tabs to lock together as the angular facets on each clamping tab are engaged; and
Cutting edges lining the openings of the spoon-shaped skin sample transporters at the distal ends of the crossed members wherein incision into desired skin sample takes place.

2. A spoon-shaped skin biopsy instrument according to claim 1 wherein the spoons-shaped biopsy instrument is used for skin excision and biopsy.

3. A spoon-shaped skin biopsy instrument according to claim 1 wherein the spoon-shaped skin biopsy instrument is used for superficial skin biopsy.
